(19) United States
(12) Patent Application Publication
(10) Pub. No.: US 2008/0257340 A1
(43) Pub. Date: Oct. 23, 2008

(54) INHALER ADAPTER

(76) Inventor: Robert Wieden, Eagan, MN (US)

Correspondence Address: INSKEEP INTELLECTUAL PROPERTY GROUP, INC, 2281 W. 190TH STREET, SUITE 200, TORRANCE, CA, 90504, US

(21) Appl. No.: 12/104,383

(22) Filed: Apr. 16, 2008

Related U.S. Application Data
(60) Provisional application No. 60/913,192, filed on Apr. 20, 2007.

(51) Int. Cl. A61M 11/00

(52) U.S. Cl. 128/200.23

(57) ABSTRACT

An adapter for use over a canister of compressed gas having an actuating valve stem is provided. The adapter redirects gas flow from the valve stem to a cavity from which the gas may be inhaled. The design of the adapter is such that the gas from the canister is transferred to the nose of a user while minimizing the amount of gas that escapes into the atmosphere prior to inhalation.

### CROSS-REFERENCE TO RELATED APPLICATIONS

This invention is related to and claims priority from U.S. provisional application Ser. No. 60/913,192 filed on Apr. 20, 2007 entitled Inhaler Adapter, which is incorporated herein by reference.

### BACKGROUND OF THE INVENTION

With the increase in interest in outdoor activities and health, and a coincidental decrease in air quality, a market has developed for pure oxygen. Breathing pure oxygen has time-established health benefits, as evidenced by its widespread use in hospitals, and is a proven way to increase alertness and athletic performance.

Heretofore, however, pure oxygen is typically administered to a user via a heavy, high-pressure tank and a face mask. The user wheels the high-pressure tank around and is encumbered thereby. If a patient requires constant oxygen, high pressure tanks are necessary in order to administer the thousands of daily breaths a patient consumes. However, in the case of an athlete or health enthusiast desiring only a few breaths as a supplement, a heavy tank is neither necessary nor desired.

Hence, a demand is growing for economical, portable bottles of oxygen. One such product is described in U.S. Pat. No. 7,178,523 to Sundhar, the contents of which are incorporated herein in its entirety. An economical container for a dispensable gas product is a standard aerosol can, such as that shown in FIG. 1. The standard can 10 includes a valve mechanism 12 that opens, releasing compressed gas, when the valve stem 14 is pressed downward or to the side. Typically, an atomizing cap is placed over the top of the can 10 and has an atomizing nozzle that fits over the valve stem 14. The cap is usually snap-fit over a lip 16 at the top edge of the can 10. The nozzle (not shown) directs the stream of gas in a desired direction. However, standard atomizing caps for aerosol cans are not designed to transfer gas from the can to the lungs of a consumer.

Moreover, most oxygen products on the market today use a mouthpiece as the breathing apparatus to inhale oxygen. This might be the easiest (and cheapest) application to incorporate into a product, but breathing through the mouth is not the most efficient way to reap the health benefits of an inhaled gas. Inhaling a gas, such as oxygen, through the mouth results in a large percentage of the gas entering the stomach instead of the lungs. Since the human stomach does not absorb gas, the gas is wasted. This is evidenced in medical applications where a patient is given either a nasal inhalation tube or a mask that covers the nose and the instruction to breathe through the nose.

It would be desirable to provide an adapter that fits over a standard compressed gas can (like an aerosol can) that is shaped to allow a user to easily inhale the contents of the can through the nose. It is also desired to provide such an adapter that minimizes the amount of gas that escapes to the atmosphere prior to being inhaled.

### SUMMARY OF THE INVENTION

The present invention provides a design for an adapter useable on a can of compressed gas for activating the valve mechanism on the can and transferring the contents of the can to the airways of a user. The adapter is designed for both user comfort and to minimize the amount of gas that escapes to the atmosphere prior to entering the airways of the user.

### BRIEF DESCRIPTION OF THE DRAWINGS

FIG. 1 is a perspective view of a can of compressed gas of the prior art;

FIG. 2 is a perspective view of an embodiment of adapter of the present invention;

FIG. 3 is a plan view of the embodiment of FIG. 2;

FIG. 4 is a section view of the embodiment of FIG. 3 taken along section lines 4-4;

FIG. 5 is a perspective view of an embodiment of adapter of the present invention;

FIG. 6 is a plan view of the embodiment of FIG. 5; and,

FIG. 7 is a section view of the embodiment of FIG. 6 taken along section lines 7-7.

### DETAILED DESCRIPTION OF THE INVENTION

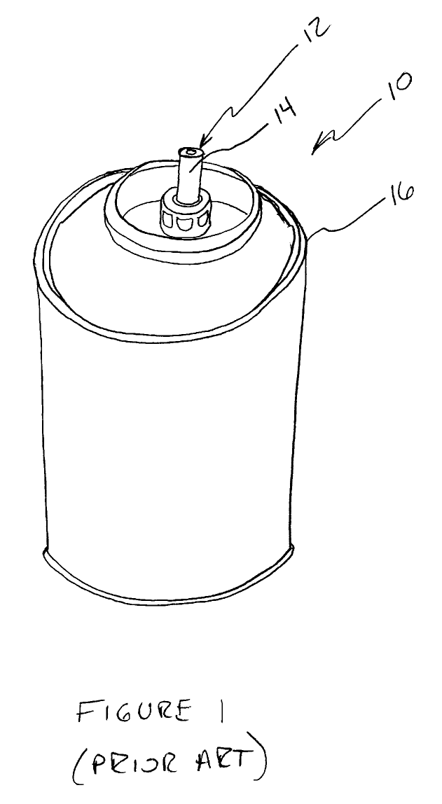

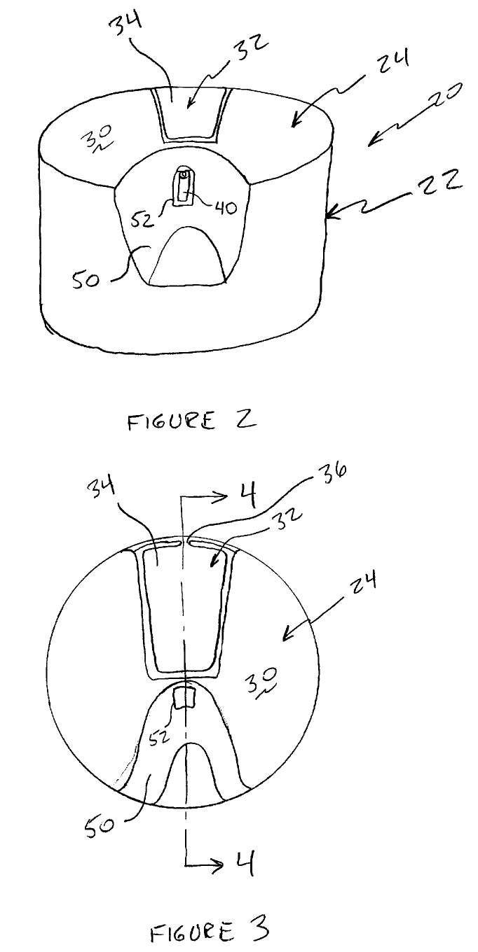

Referring now to the figures and first to FIGS. 2-4, there is shown one embodiment of an adapter 20 of the present invention. The adapter 20 generally includes a sidewall 22 and a top 24. The sidewall 22 is generally cylindrical and sized to fit over the end of a can of compressed gas, an example 10 of which is shown in FIG. 1. The sidewall 22 may include one or more projections 26 extending inwardly from an inside surface 28 of the sidewall 22 to form a snap-fit over the lip 16 of the can 10.

The top 24 includes an upper surface 30 and an actuator 32. The actuator 32 is a lever used to actuate the valve stem 14 of a can 10. The actuator 32 includes a push button 34 that pivots downwardly around a connector 36 that connects the actuator 32 to the rest of the adapter 20. The push button 34 has a nozzle 40 that is sized and located to fit over a valve stem 14 when the adapter 20 is snap-fit onto a can 10.

A cavity 50 is formed in the sidewall 22 and the top 24. The cavity 50 is sized and shaped to receive a user's nose and includes a hole 52 that leads to an interior 42 of the adapter 20. The nozzle 40 is shaped to redirect an upward stream of gas emitted from a can 10 through the hole 52 and into the cavity 50 where it can be inhaled by a user. The cavity 50 is preferably shaped to minimize the amount of gas that escapes into the atmosphere before being inhaled by the user.

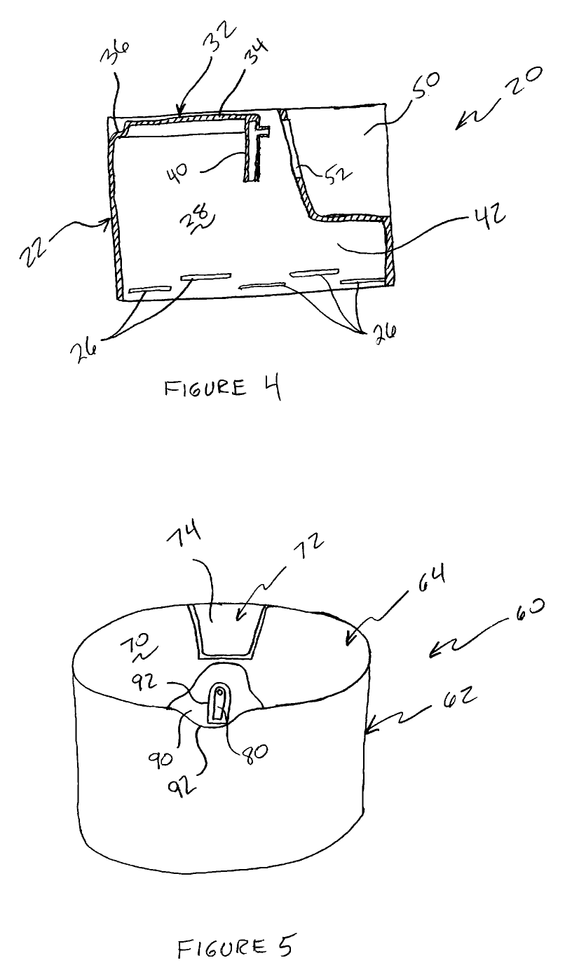

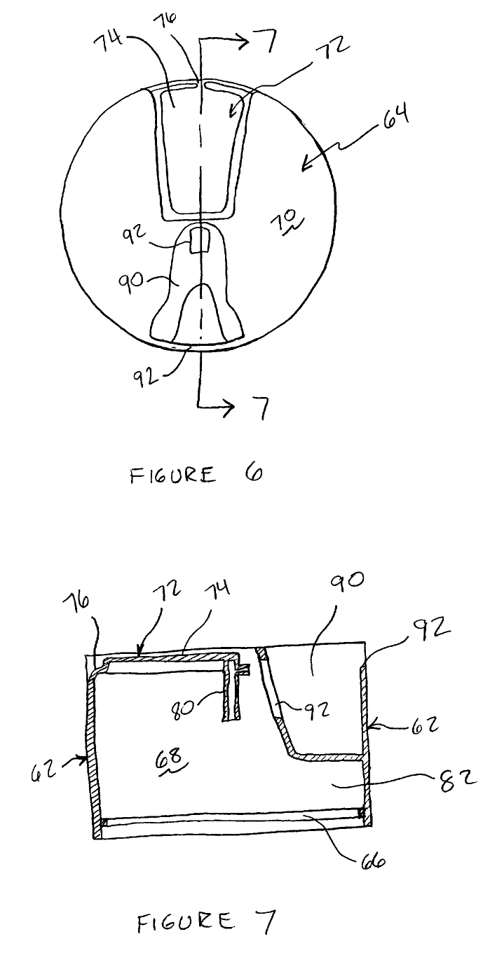

FIGS. 5-7 show an embodiment 60 of the adapter of the present invention. Adapter 60 is similar to adapter 20 except that a different cavity design is provided to minimize the escape of gas to the atmosphere prior to inhalation. As such, the adapter 60 generally includes a sidewall 62 and a top 64. The sidewall 62 is generally cylindrical and sized to fit over the end of a can of compressed gas, an example 10 of which is shown in FIG. 1. The sidewall 62 may include one or more projections 66 extending inwardly from an inside surface 68 of the sidewall 62 to form a snap-fit over the lip 16 of the can 10.

The top 64 includes an upper surface 70 and an actuator 72. The actuator 72 is a lever used to actuate the valve stem 14 of a can 10. The actuator 72 includes a push button 74 that pivots downwardly around a connector 76 that connects the actuator 72 to the rest of the adapter 60. The push button 74 has a nozzle 80 that is sized and located to fit over a valve stem 14 when the adapter 60 is snap-fit onto a can 10.

A cavity 90 is formed in the sidewall 62 and the top 64. The cavity 90 is sized and shaped to receive a user's nose and includes a hole 92 that leads to an interior 82 of the adapter 60. The nozzle 80 is shaped to redirect an upward stream of gas emitted from a can 10 through the hole 92 and into the cavity 90 where it can be inhaled by a user. The cavity 90 is preferably shaped to minimize the amount of gas that escapes into the atmosphere before being inhaled by the user. As such, the sidewall 62 extends upward to form a substantially enclosed cavity 90 with an open top over a user can place his or her nose. Hence, the user's nose completely encloses the cavity 90, thereby ensuring that substantially all of the gas gets inhaled by the user. A small recess 92 in the sidewall 62 may be provided to allow room for the user's septum.

Both of the embodiments 20 and 60 have tops that are flat. This is preferable for purposes of stacking and if a liquid container is to be attached to an opposite end of the can 10. In this case, the adapter serves a second function of a platform for setting the beverage/gas combination canister down on a flat surface. Furthermore, it is preferable, though not required, that the embodiments 20 and 60 be of unitary construction, for economical manufacturing purposes.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An inhaler adapter comprising:
a sidewall;
a top attached to said sidewall;
a cavity defined by said sidewall and said top;
an actuator pivotally attached to one of said sidewall and said top and independent of the other of said sidewall and said top, said actuator including a nozzle constructed and arranged to receive a gas from a container of pressurized gas and direct said gas into said cavity.

2. The adaptor of claim 1 wherein said sidewall is shaped and sized to fit over a can of compressed air such that said nozzle is in fluid communication with a valve stem of said can.

3. The adaptor of claim 1 wherein said sidewall forms a snap-fit over a lip of a can of compressed gas.

4. The adaptor of claim 1 wherein said sidewall includes at least one projection extending inward from an inside surface of said sidewall.

5. The adapter of claim 1 wherein said cavity definition comprises an opening in said top that is continuous with an opening in said sidewall.

6. The adapter of claim 5 wherein said opening in said sidewall extends from said top to a bottom of said cavity.

7. The adapter of claim 1 wherein said cavity definition comprises an opening in said top and wherein said cavity is radially defined by said sidewall.

8. The adapter of claim 7 wherein said sidewall comprises a recess, proximate said cavity, for accommodating a user's septum.

9. The adaptor of claim 1 wherein said top is flat.

10. The adaptor of claim 1 wherein when said adaptor is coupled to a can of compressed air said top surface may be used as a platform, when inverted, for supporting said can on a flat surface.

11. The adaptor of claim 9 wherein said top supports said can when said can is in an up-side-down position relative to said flat surface.

12. The adaptor of claim 1 wherein said cavity is formed at an intersection of said sidewall and said top.

13. The adaptor of claim 1 wherein said cavity comprises a hole that leads to an interior of said adaptor.

14. The adaptor of claim 1 wherein said cavity is selectively sized and shaped to receive a user's nose.

15. The adaptor of claim 1 wherein said cavity is selectively sized and shaped to minimize the escape of gas during a user's inhalation.

16. A method for directing gas from a can of compressed gas to a nose of a user comprising:
attaching an adaptor to a can of compressed gas;
receiving nose in a cavity formed in said adaptor, and depressing an actuator of said adaptor, wherein said actuator opens a valve stem on said can of compressed gas releasing said compressed gas;
directing said released gas into said cavity where said gas may be inhaled by said user.
